(19) United States
(12) Patent Application Publication
(10) Pub. No.: US 2025/0302576 A1
(43) Pub. Date: Oct. 2, 2025

(54) BIOPSY SITE MARKER WIRELESS-POWERED VIBRATION

(71) Applicant: Devicor Medical Products, Inc., Cincinnati, OH (US)

(72) Inventors: James O. ROGERS, III, Milford, OH (US); David R. SANDER, Lebanon, OH (US); Lyle JAMES, Kettering, OH (US); Spencer CHAMBERLAIN, Cleves, OH (US); Eric J. MILLER, Cincinnati, OH (US); Kyle WAGNER, Mason, OH (US)

(21) Appl. No.: 19/042,233

(22) Filed: Jan. 31, 2025

Related U.S. Application Data
(60) Provisional application No. 63/570,301, filed on Mar. 27, 2024.

(51) Int. Cl. A61B 90/00; A61B 10/00

(52) U.S. Cl. CPC A61B 90/39; A61B 10/0041; A61B 2090/3908; A61B 2090/3929; A61B 2090/3987

(57) ABSTRACT

A biopsy site marker includes a carrier, a marker element, and a locator. At least a portion of the marker element is disposed within a portion of the carrier. The locator is configured to emit vibrations into tissue proximate the biopsy site marker.

### PRIORITY

This application claims priority to U.S. Provisional Application Ser. No. 63/570,301, entitled “Biopsy Site Marker Wireless-Powered Vibration,” filed on Mar. 27, 2024, the disclosure of which is incorporated by reference herein.

### BACKGROUND

A number of patients will have breast biopsies because of irregular mammograms and palpable abnormalities. Biopsies can include surgical excisional biopsies and stereotactic and ultrasound guided needle breast biopsies. In the case of image directed biopsy, the radiologist or other physician may take a small sample of tissue for laboratory analysis. If the biopsy proves to be malignant, additional surgery (e.g., a lumpectomy or a mastectomy) may be required. In the case of needle biopsies, the patient may return to the radiologist a day or more later, and the biopsy site (the site of the lesion) may need to be relocated in preparation for the surgery. An imaging system, such as ultrasound, magnetic resonance imaging (MRI) or x-ray may be used to locate the biopsy site. In order to assist the relocation of the biopsy site, a marker may be placed at the time of the biopsy.

The use of markers after breast biopsies to mark the location where the biopsied tissue was removed is described in the following US patents: U.S. Pat. No. 6,083,524, “Polymerizable biodegradable polymers including carbonate or dioxanone linkages,” issued Jul. 4, 2000; U.S. Pat. No. 6,162,241, “Hemostatic tissue sealants,” issued Dec. 4, 2000; U.S. Pat. No. 6,270,464, “Biopsy localization method and device,” issued Aug. 7, 2001; U.S. Pat. No. 6,356,782, “Subcutaneous cavity marking device and method,” issued Mar. 12, 2002; U.S. Pat. No. 6,605,294, “Methods of using in situ hydration of hydrogel articles for sealing or augmentation of tissue or vessels,” issued Aug. 12, 2003; U.S. Pat. No. 8,600,481, “Subcutaneous cavity marking device,” issued Dec. 3, 2013 and U.S. Pat. No. 8,939,910, “Method for enhancing ultrasound visibility of hyperechoic materials”, issued Jan. 27, 2015. All of these US patents are incorporated by reference in their entirety.

In some circumstances, a marker is located with the use of ultrasound or x-ray technology. Such location methods may have challenges due to the need for additional equipment. Additionally, such location methods may be limited by particular attributes of the marker itself or surrounding tissue. In other circumstances, markers using elongate localization wires may be used. Such localization wires may be configured to protrude from the patient's skin to pinpoint the location of the marker. The protruding localization wire may then be traced to the location of the marker corresponding to the biopsy site. However, such wire localization methods may be inconvenient or uncomfortable for the patient in certain circumstances.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements.

FIG. 1 depicts a perspective view of an illustrative biopsy site marker;

FIG. 2 depicts a perspective cross-sectional view of the biopsy site marker of FIG. 1;

FIG. 3 depicts a perspective view of an illustrative marker delivery device for use with the biopsy site marker of FIG. 1;

FIG. 4A depicts a perspective view of the biopsy site marker of FIG. 1 being deployed from the marker delivery device of FIG. 3 and through a lateral aperture in a biopsy needle to mark a biopsy site;

FIG. 4B depicts a perspective view of the biopsy site marker of FIG. 1 after being deployed from the marker delivery device of FIG. 3 and through a lateral aperture in a biopsy needle to mark a biopsy site;

FIG. 5 depicts a perspective view of the biopsy site marker of FIG. 1 after being deployed, at least a portion of the biopsy site marker being activated using an ultrasonic transducer; and

FIG. 6 depicts a perspective view of an illustrative alternative biopsy site marker for use with the marker delivery device of FIG. 3.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

It may be beneficial to be able to mark the location or margins of a lesion, whether temporarily or permanently, prior to or immediately after removing or sampling it. Marking prior to removal may help to ensure that the entire lesion is excised, if desired. Alternatively, if the lesion were inadvertently removed in its entirety, marking the biopsy site immediately after the procedure would enable reestablishment of its location for future identification. Once a marker is positioned in a biopsy site, it may be desirable for the marker to remain visible. For instance, it may be desirable for the marker to produce a signal such as vibration, light, radio frequency or other signals configured to penetrate tissue for enhanced localization of the marker either through direct visualization, palpitation, or under image guidance (e.g., ultrasound).

### I. EXAMPLE OF BIOPSY SITE MARKER WITH SIGNAL EMISSION

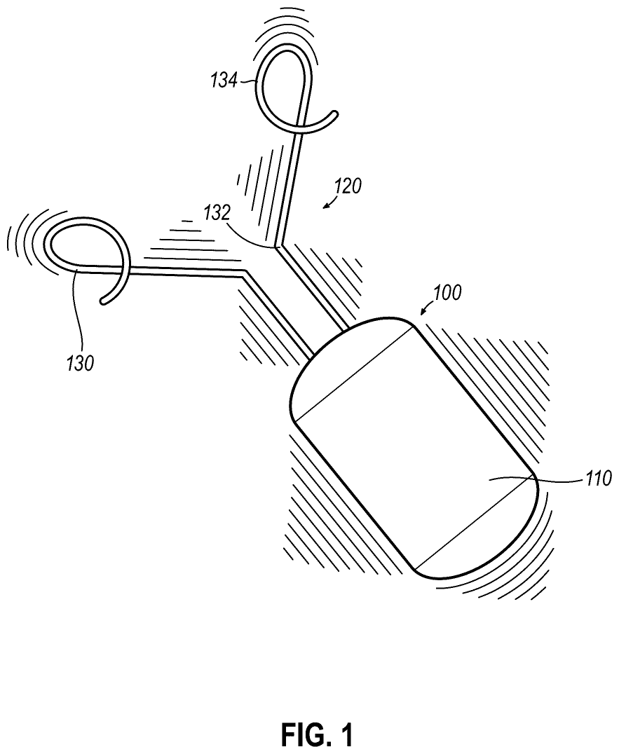

FIG. 1 shows an illustrative biopsy site marker (100) that is generally configured for deployment in tissue to mark the location of a biopsy site (100). As will be described in greater detail below, one or more portions of marker (100) are further configured to emit a signal such as vibration to facilitate localization of marker (100) within tissue. Marker (100) of the present example includes a carrier (110), a marker element (120), and a locator (150).

Carrier (110) defines at least a portion of the outer surface of marker (100). In other words, carrier (110) is generally configured to enclose one or more other features of marker (100), as will be described in greater detail below. In the present example, carrier (110) is configured as a bioabsorbable component, which may be absorbed into tissue over time once marker (100) is deployed at a biopsy site. Optionally, carrier (110) is configured to expand in some examples. Such expansion can be facilitated by absorption of fluid adjacent to carrier (110) from surrounding tissue. In some examples, expansion properties may be desirable to fill a biopsy cavity to fix the location of marker (100) within tissue. By way of example only, suitable materials for carrier (110) may include hydrogels, other polymers, and/or collagen.

In some examples, carrier (110) may also be configured to enhance localization of marker (100) in some circumstances. For instance, in examples where carrier (110) is configured to absorb fluid, such fluid absorption may additionally facilitate localization of marker under ultrasound by providing a difference in density between tissue and carrier (110). Such a difference in density may reflect ultrasonic radiation at the interface between carrier (110) and tissue. In addition, or in the alternative, carrier (110) can optionally include other elements to facilitate localization of marker (100) under ultrasound such as air bubbles, microspheres, or other reflective agents.

Marker element (120) is generally configured to facilitate localization of marker (100). For instance, marker element (120) is generally configured as a hard non-bioabsorbable (e.g., permanent or semi-permanent) material with radiopaque and/or echogenic properties to enhance visualization of marker (100) over relatively long durations (e.g., months or years). Thus, marker element (120) is generally formed into a variety of different shapes to enhance visualization of marker (100) under ultrasound, x-ray, or both.

In the present example, at least a portion of marker element (120) is disposed or embedded within carrier (110). As will be described in greater detail below, a portion of marker element (120) may optionally extend from carrier (110). In other examples, carrier (110) may be omitted entirely and marker (100) may be configured as a “bare” marker with marker element being entirely exposed and not embedded within a structure such as carrier (110) described above.

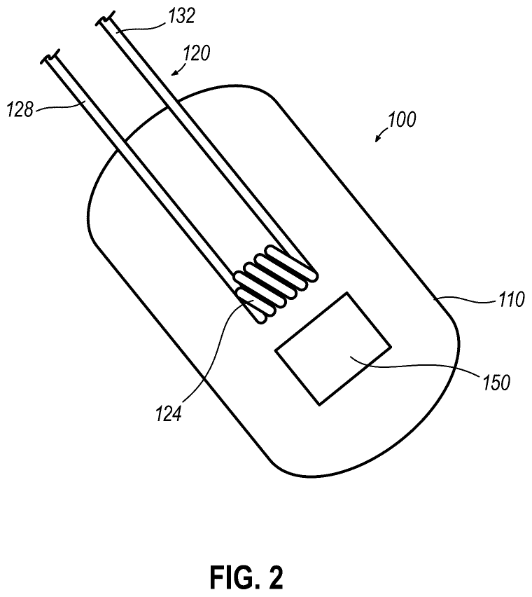

Optionally, marker element (120) may be configured to prevent migration of marker (100) or otherwise mechanically ground marker (100) in some examples. For instance, as best seen in FIGS. 1 and 2, marker element (120) of the present example includes a base (124) and a plurality of legs (128, 132) extending away from base (124). Base (124) defines a generally coil-shaped configuration, which is configured to drive legs (128, 132) outwardly and into tissue. Meanwhile, legs (128, 132) each include a respective loop (130, 134) disposed outside of carrier (110), which may be configured to grip or otherwise engage tissue.

In some examples, the coil structure of base (124) is also configured to facilitate visualization under ultrasound and/or x-ray. For instance, the coiled shape generally provides a plurality of surfaces, which may reflect ultrasonic radiation regardless of the orientation of base (124) relative to an ultrasonic source. Similarly, the irregular structure of the coil shape, may provide a distinctive appearance of base (124) under x-ray, regardless of the orientation of the x-ray source and detector relative to base (124). Although a coiled structure is used in the present example for base (124), it should be understood that other suitable structures may be used in other examples such as twisted plate structures, twisted wire structures, solid barbell shapes, and/or etc. In some examples, one or more features of marker element (120) are configured in accordance with the teachings of U.S. Pat. No. 10,842,591, entitled “Biopsy Marker with Anchoring Capabilities,” issued on Nov. 24, 2020, the disclosure of which is hereby incorporated by reference herein.

Because base (124) provides both functions related to anchoring of marker (100) and functions related to visualization of marker (100), it should be understood that marker element (120) may be modified in some examples to eliminate one or more functions of base (124). For instance, in some examples, structures such as legs (128, 132) are omitted such that base (124) is only used for visualization related functions. In other words, in some examples, marker element (120) can comprise only base (124) with a coil shape or any other suitable shape described herein.

As best seen in FIG. 2, marker (100) of the present example further includes locator (150) with at least a portion of locator (150) being disposed within carrier (110). Locator (150) is generally configured to emit a signal to facilitate enhanced localization of marker (100). For instance, in the present example, locator (150) is configured to emit vibrations, which may create disturbances in an ultrasonic field to provide a distinctive appearance under ultrasound. During such vibrations, marker (100) generally remains in a fixed position within tissue, while at least a portion of marker (100) also oscillates about an equilibrium point. In other examples, locator (150) may be configured to emit a variety of other signals either in addition to, or in lieu of, vibrations such as infrared and/or visible light, and/or electromagnetic radiation.

In the present example, locator (150) includes a piezoelectric crystal configured to be excited in the presence of ultrasonic radiation. In response, the piezoelectric crystal is configured to resonate to produce vibration. In some examples, the piezoelectric crystal is configured to resonate at a predetermined frequency. Such frequencies may correspond to frequencies for enhanced visualization under ultrasound. Suitable vibrations may include low frequencies in perceptible to humans. Still other suitable vibrations may include high frequencies. Suitable high frequencies may include about 27,000,000 hertz (27 Megahertz). Yet other suitable high frequencies may include 26,700,000 hertz to 30,000,000 hertz. Still other suitable high frequencies may include 25,000,000 hertz to 30,000,000 hertz. Still other suitable high frequencies will be apparent to those of ordinary skill in the art in view of the teachings herein.

Although locator (150) of the present example includes a piezoelectric crystal to produce vibration, it should be understood that other component may be used in other examples either in addition to, or in lieu of, the piezoelectric crystal. For instance, in some examples, locator (150) may include a micro-electromechanical system (MEMS) motor configured to drive a vibrator or oscillator. In addition, or in the alternative, locator (150) may include one or more magnetic vibration generators, one or more EMF-based vibration generators, and/or combinations thereof. In such examples, one or more components of locator (150) may be driven via a separate power source such as one or more batteries, one or more thermoelectric generators, and/or one or more kinetic generators. Alternatively, in some examples, a piezoelectric crystal may generate electric current using ultrasonic radiation used during ultrasonic imaging, which may in turn power other components of locator (150).

Locator (150) may be in a variety of positions relative to marker element (120) and or carrier (110). For instance, in the present example, locator (150) is disposed within carrier (110) proximate base (124) of marker element (120). In other examples, locator (150) is disposed within carrier (110) between legs (128, 132). In still other examples, such as examples of marker (100) without carrier (110), locator (150) is positioned outside of carrier (110) proximate a portion of marker element (120).

Optionally, locator (150) is in communication with a portion of marker element (120). Such communication may be desirable to transmit vibrations or other signals from locator (150) to other portions of marker (100). For instance, in some examples, locator (150) is configured to transmit vibrations or other signals through marker element (120) and through legs (128, 134) to provide broader communication of such vibrations. In some examples, such transmission is further facilitated by integration of at least a portion of locator (150) into marker element (120). For instance, locator (150) may be positioned between one or more coils defined by base (124). Such a configuration may be desirable to both promote transmission of vibrations and/or other signals and to secure the position of locator (150) relative to marker element (120) (particularly in versions of marker (100) without carrier (110)).

### II. EXAMPLE OF MARKER DELIVERY DEVICE

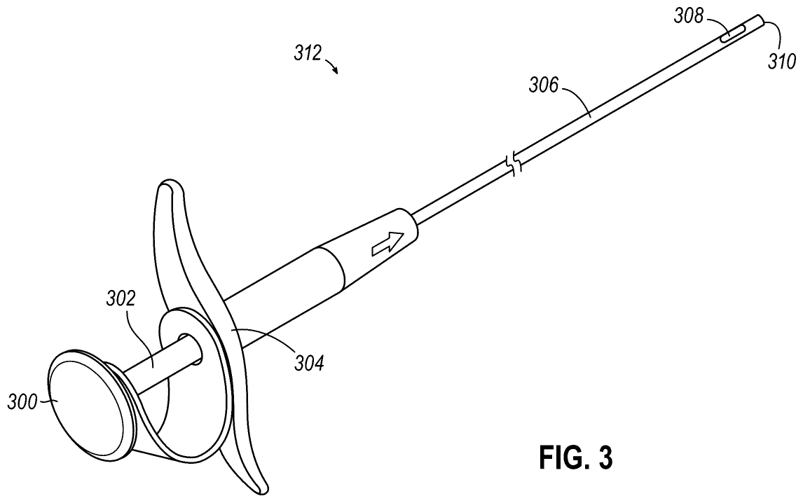

In some circumstances it may be desirable to deploy marker (100) described above within the body cavity using certain marker delivery devices. For instance, FIG. 3 shows an illustrative marker delivery device (312) which includes an elongate outer cannula (306) having a marker exit, such as side opening (308) formed adjacent to, but spaced proximally from, a distal end (310) of the cannula (306).

A grip (304) can be provided at the proximal end of cannula (306). A push rod (302) can be provided, with push rod (302) extending coaxially in cannula (306) such that push rod (302) is configured to translate within cannula (306) to displace one or more markers through side opening (308). Rod (302) may have sufficient rigidity in compression to push a marker from an internal lumen of cannula (306) out through side opening (308), yet be relatively flexible in bending. A plunger (300) is coupled at the proximal end of rod (302) for forcing rod (302) distally in cannula (306) to deploy a marker out of cannula (306).

A user may grasp grip (304) with two fingers, and may push on plunger (300) using the thumb on the same hand, so that marker delivery device (312) is operated by a user's single hand. A spring (not shown) or other feature may be provided about rod (302) to bias rod (302) proximally relative to grip (304) and cannula (306).

Cannula (306) may be formed of any suitable metallic or non-metallic material. In some versions, cannula (306) is formed of a thin-walled hollow tube formed of a suitable medical grade plastic or polymer. One suitable material is a thermoplastic elastomer, such as Polyether block amide (PEBA), such as is known under the tradename PEBAX. Cannula (306) may be formed of PEBAX, and may be substantially transparent to visible light and X-ray.

Side opening (308) may be formed by cutting away a portion of the wall of cannula (306). Side opening (308) communicates with an internal lumen of cannula (306). Side opening (308) may extend axially (in a direction parallel to the axis of lumen) from a proximal opening end to a distal opening end. In some versions, side opening (308) may be configured as an opening in the distal end of cannula (306).

### III. EXAMPLE USE OF BIOPSY SITE MARKER

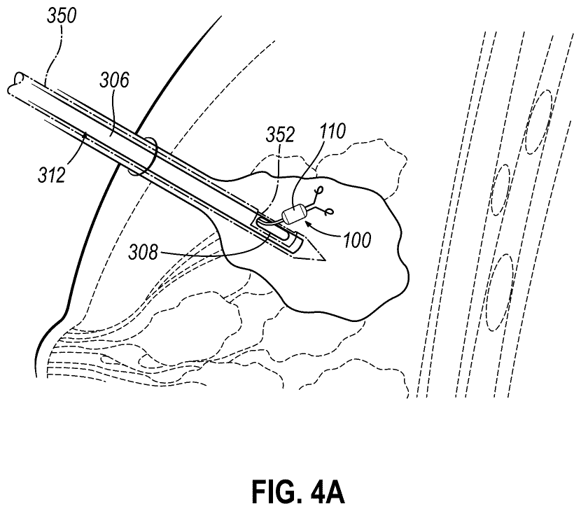

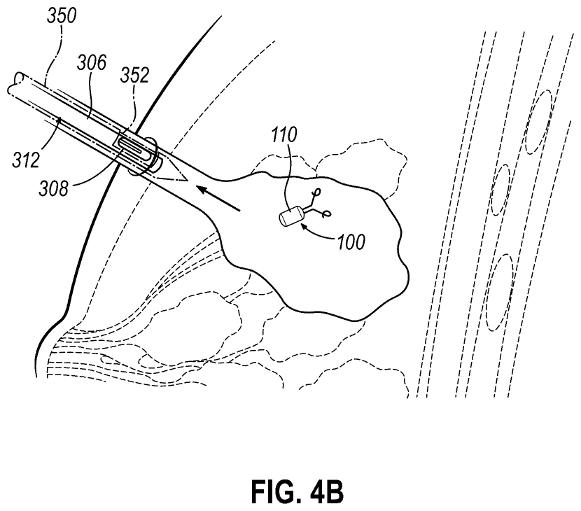

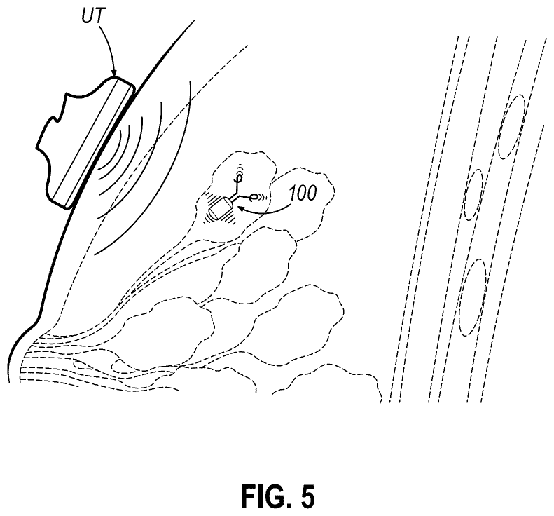

FIGS. 4A through 5 show an example procedure for use of biopsy site marker (100) described above. As best seen in FIG. 4A, marker (100) is initially disposed within outer cannula (306) of marker delivery device (312) for deployment at a biopsy site. Optionally, outer cannula (306) is inserted through a biopsy needle (350) to access the biopsy site through a lateral aperture (352) defined by a portion of needle (350). Alternatively, outer cannula (306) itself may be inserted into tissue and guided to the biopsy site under various modes of image guidance (e.g., ultrasound, x-ray, etc.).

Regardless, once outer cannula (306) is positioned at the biopsy site, marker (100) is deployed though side opening (308) of outer cannula (306) using push rod (302). Once marker (100) is deployed, outer cannula (306), needle (350), and/or etc. are removed from the patient leaving marker (100) in place as seen in FIG. 4B. At this stage, legs (128, 132) of marker element (120) may move to engage tissue. Additionally, carrier (110) may absorb moisture from surrounding tissue to expand and thereby fill a cavity formed at the biopsy site. With marker (100) deployed, the tissue surrounding the biopsy site can be closed.

After deployment, it may be desirable to relocate the biopsy site using marker (100). Relocation may occur at a variety of intervals between deployment of marker (100) and relocation. In some examples, relocation may occur days, weeks, months, or years after deployment of marker (100). Regardless, relocation may be performed using ultrasonic imaging. In some examples, ultrasonic imaging may be facilitated by a system including a controller, display, and one or more ultrasound transducers (UT). Generally, the ultrasound transducer (UT) is configured to emit and receive ultrasonic radiation, which can be processed by the controller to graphically depict an area near the ultrasound transducer (UT) and detect the presence of certain objects such as marker (100).

As shown in FIG. 5, ultrasound transducer (UT) is moved on the surface of the patient's skin to direct ultrasound radiation through tissue towards marker (100). Some ultrasound radiation may propagate through marker (100) and into contact with locator (150). The ultrasound radiation then excites one or portions of locator (150), resulting in the one or more portions (e.g., piezoelectric crystals) producing vibrations as a function of the frequency of the ultrasound radiation. The vibrations may then interact with subsequent ultrasonic radiation returning to ultrasound transducer (UT). Such interaction may be detectable on the display of the system making marker (100) more readily identifiable.

Although the present use uses ultrasonic radiation from ultrasound transducer (UT) to generate a signal via locator (150), in other uses, locator (150) is configured to generate a signal independently of ultrasound transducer (UT). In such uses, locator (150) can be configured to generate vibrations or other signals continuously without regard to the presence of ultrasonic radiation via ultrasound transducer (UT). In other uses, locator (150) can be configured to generate vibrations or other signals selectively and may be activated by the presence of ultrasonic radiation from ultrasound transducer (UT) or other suitable means of activation (e.g., an electromagnetic field, palpation, etc.). In still other examples, locator (150) can be configured to generate vibrations or other signals intermittently.

### IV. ALTERNATIVE EXAMPLE OF BIOPSY SITE MARKER WITH SIGNAL EMISSION

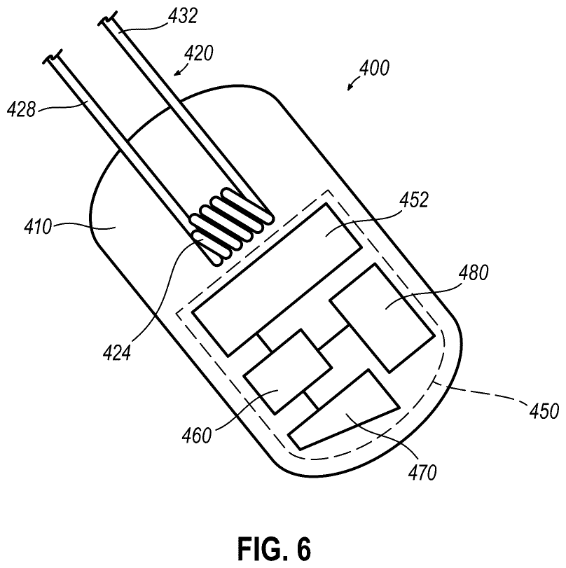

FIG. 6 shows an illustrative alternative biopsy site marker (400), which may be used to mark a biopsy site either in addition to, or in lieu of, biopsy site marker (100) described above. Marker (400) of the present example is substantially similar to marker (100) described above. For instance, like marker (100) described above, marker (400) of the present example includes a permanent or semi-permanent marker element (420) fully or partially enclosed within a temporary bioabsorbable carrier (410). As similarly described above with respect to carrier (110), carrier (410) is generally configured to expand in the presence of moisture to fill a biopsy cavity. As also similarly described above, carrier (410) is configured for absorption into tissue over time and may include certain features to improve visibility of carrier (410) such as microspheres, air bubbles, and/or etc. To facilitate such functionality, carrier (410) can include hydrogel, collagen, polymers, and/or other suitable materials.

Marker element (420) of the present example is substantially similar to marker element (120) described above. For instance, like marker element (120) described above, marker element (420) of the present example includes a base (424) defining a coil-shaped structure. As similarly described above, base (424) can be configured to provide enhanced visibility under ultrasound and or x-ray imaging using the coil-shaped structure or other suitable shaped structures.

Additionally, marker element (420) optionally further includes one or more legs (428, 432), which may be used to anchor marker (400) in tissue. As similarly described above, legs (428, 432) extend from base (424) and further extend from a portion of carrier (410). In some examples, legs (428, 432) are configured to move via the coil shape of base (424) and/or expansion of carrier (410) to drive engagement between legs (428, 432) and tissue. Although not shown it should be understood that legs (428, 432) may optionally include one or more loops similar to loops (130, 134) described above.

Like marker (100) described above, marker (400) of the present example also includes a locator (450). However, unlike locator (150) described above, locator (450) of the present example includes a plurality of independent elements to separate the functions of receiving power and emitting vibrations and/or other signals. Such a configuration may be desirable in some circumstances to provide enhanced functionality such as the use of multiple signal types, selectable signal types, selectable signal patterns, multiple power communication modes, and/or etc.

Locator (450) of the present example includes a signal emitter (452), controller (460), receiver (470), and a power source (480). Controller (460) is in communication with signal emitter (452), receiver (470) and power source (480) to control input and output functions associated with such elements. In some examples, controller (460) may include integrated circuits, processing units, memory units, and/or etc. to provide flexible control of signal emitter (452), receiver (470) and/or power source (480). In still other examples, controller (460) can include simple control circuitry to provide pre-determined control of signal emitter (452), receiver (470), and/or power source (480).

Signal emitter (452) is generally configured to emit one or more signals, which may be used to locate marker (400) within tissue. Similarly to locator (150) described above, signal emitter (452) of the present example is disposed within a portion of carrier (410) proximate a portion of marker element (420) such as base (424). In some examples, at least a portion of signal emitter (452) is incorporated into a portion of marker element (420) such as base (424). As similarly described above, such a configuration may be desirable to secure signal emitter (452) (e.g., for versions with or without carrier (410)) and/or propagate one or more signals via marker element (420).

Signal emitter (452) can include a variety of emitters either separately or in combination. For instance, in some examples, signal emitter (452) can include one or more piezoelectric crystals or other vibratory elements, which can be configured to emit vibrations. In other examples, signal emitter (452) can include light emitters either in combination with piezoelectric crystals or in lieu of piezoelectric crystals. In such examples, light emitters are configured to emit light of the visible or non-visible spectra including, for example, visible light and/or infrared light. In still other examples, signal emitter (452) can include one or more radio frequency emitters either in combination with piezoelectric crystals and light emitters or in lieu of piezoelectric crystals and/or light emitters.

Receiver (470) is generally configured to receive power from a remote power source or otherwise obtain or harvest power (e.g., from body heat and/or body motion). In some examples, receiver (470) is configured to supply power to power source (480), which may be used to power signal emitter (452). In other examples, receiver (470) is configured to power signal emitter directly (452). In such examples, power source (480) and controller (460) may be omitted. Thus, it should be understood that power source (480) and controller (460) are optional in the present example.

Receiver (470) can be configured to receive power through a variety of modes. For instance, in some examples, receiver (470) is configured to receive power via ultrasonic radiation. In such examples, receiver (470) includes a piezoelectric crystal configured to vibrate in the presence of ultrasonic radiation. This vibration can then result in the piezoelectric crystal generating an electric current. In other examples, receiver (470) is configured to receive power via electromagnetic radiation. In such examples, receiver (470) includes a coil of wire. When the coil of wire is positioned within an electromagnetic field emitted by a remote source, the electromagnetic field may induce a current in the coil of wire. In yet other examples, receiver (470) includes a combination of elements such as piezoelectric crystals and coils of wire to receive power through a combination of different modes. In still other examples, receiver (470) includes element configured to draw power from bodily tissues either through body heat or movement of the body.

Power source (480) is configured to receive and store power received by receiver (470). As described above, power source (480) is optional because receiver (470) is configured to power signal emitter (452) directly in some examples. Alternatively, in some examples, power source (480) may be pre-charged with a predetermined amount of power, thus making receiver (470) optional. Power source (480) may include a variety of elements configured to store power. By way of example only, power source (480) can include one or more batteries, one or more capacitors, and/or etc.

In use, marker (400) can be deployed at a biopsy site using marker deliver device (312) as similarly described above with respect to marker (100). Once marker (400) is deployed at the biopsy site, locator (450) can be used in a variety of ways to enhance viability of marker (400) under ultrasound, x-ray imaging and/or visual perception without imaging guidance. For instance, under ultrasound visualization, the ultrasonic radiation associated with the ultrasound visualization may be used by receiver (470) to power source (480) or signal emitter (452) directly. Controller (460) may then be used to control signal emitter (452) to emit one or more signals via signal emitter (452). Such signals can include vibrations, visible light, infrared light, radio frequencies, electromagnetic fields, and/or etc. In some uses, controller (460) can control signal emitter (452) to emit such signals continuously, only in the presence of ultrasonic radiation, intermittently, or on a predetermined schedule. In other uses, signa emitter (452) is powered directly by receiver (470) and therefore may be configured to emit such signals only in the presence of ultrasonic radiation. In yet other uses, receiver (470) is configured to receive power separately from ultrasonic radiation present during ultrasonic imaging. In such uses, a separate power emitter can be used to transmit power to receiver (470), which may be used to power signal emitter (452).

### V. EXAMPLE COMBINATIONS

### Example 1

A biopsy site marker, the marker comprising: a carrier; a marker element, at least a portion of the marker element being disposed within a portion of the carrier; and a locator, the locator being configured to emit vibrations into tissue proximate the marker.

### Example 2

The marker of Example 1, the locator being in communication with the marker element.

### Example 3

The marker of Examples 1 or 2, the marker element including a base defining a coil-shaped structure, the locator being disposed within the coil-shaped structure of the base.

### Example 4

The marker of Examples 1 or 2, the locator defining at least a portion of the marker element.

### Example 5

The marker of any of Examples 1 through 4, the locator including a signal emitter and a power receiver, the power receiver being in communication with the signal emitter to emit vibrations via the signal emitter.

### Example 6

The marker of Example 5, the locator further including a controller, the controller being in communication with the signal emitter and the power receiver, the controller being configured to drive the signal emitter to emit vibrations in a predetermined sequence.

### Example 7

The marker of Examples 5 or 6, the locator further including a power source, the power source being configured to store power received via the power receiver and communicate the stored power to the signal emitter.

### Example 8

The marker of any of Examples 1 through 7, the locator including a piezoelectric crystal.

### Example 9

The marker of any of Examples 1 through 8, the locator being configured to emit vibrations in the presence of ultrasonic radiation.

### Example 10

The marker of any of Examples 1 through 8, the locator being configured to emit vibrations continuously in the presence of ultrasonic radiation.

### Example 11

The marker of any of Examples 7 through 10, the locator being configured to emit vibrations imperceptible to humans.

### Example 12

The marker of any of Examples 1 through 11, the locator being configured to emit vibrations of 25 Megahertz to 30 Megahertz.

### Example 13

The marker of any of Examples 1 through 12, the locator being entirely enclosed by the carrier.

### Example 14

The marker of any of Examples 1 through 13, the marker element including one or more legs, each leg of the one or more legs extending from an outer surface of the carrier, the locator being configured to communicate the vibrations through at least one leg of the one or more legs.

### Example 15

The marker of any of Examples 1 through 14, the carrier being bioabsorbable, the marker element and the locator being permanent.

### Example 16

A system for locating a biopsy site, the system comprising: an ultrasound transducer; and a biopsy site marker, the marker including: a marker element, and a locator proximate the marker element, the ultrasound transducer being configured to transmit ultrasonic radiation through tissue, the locator being configured to be excited by the ultrasonic radiation transmitted by the ultrasound transducer, thereby producing vibrations.

### Example 17

The system of Example 16, the locator being configured to resonate with the ultrasonic radiation transmitted by the ultrasound transducer.

### Example 18

The system of Examples 16 or 17, the locator being coupled to a portion of the marker element, the marker element being configured to transmit the vibrations produced by the locator.

### Example 19

The system of any of Examples 16 through 18, the marker further including a bioabsorbable carrier, the carrier enclosing the locator and at least a portion of the marker element.

### Example 20

A method for locating a biopsy site, the method comprising: inserting a portion of a marker delivery device into tissue to position a deployment opening of a cannula of the marker delivery device at the biopsy site; actuating a push rod of the marker delivery device to project a biopsy site marker through the deployment opening of the cannula of the marker delivery device and into the biopsy site; and inducing a vibration from at least a portion of the marker.

### Example 21

The method of Example 20, the step of inducing the vibration from at least a portion of the marker including communicating ultrasonic radiation toward the marker to induce the vibration.

### Example 22

The method of Examples 20 or 21, further comprising visualizing the vibration induced in the at least a portion of the marker using ultrasonic imaging guidance.

### Example 23

The method of any of Examples 20 through 22, further comprising reflecting ultrasonic radiation from one or more of a marker element, a carrier, and a locator of the marker, the reflection of the ultrasonic radiation producing a distinct signal from the vibration diced in the at least a portion of the marker.

### VI. CONCLUSION

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. A biopsy site marker, the marker comprising:
(a) a carrier;
(b) a marker element, at least a portion of the marker element being disposed within a portion of the carrier; and
(c) a locator, the locator being configured to emit vibrations into tissue proximate the marker.

2. The marker of claim 1, the locator being in communication with the marker element.

3. The marker of claim 1, the marker element including a base defining a coil-shaped structure, the locator being disposed within the coil-shaped structure of the base.

4. The marker of claim 1, the locator defining at least a portion of the marker element.

5. The marker of claim 1, the locator including a signal emitter and a power receiver, the power receiver being in communication with the signal emitter to emit vibrations via the signal emitter.

6. The marker of claim 5, the locator further including a controller, the controller being in communication with the signal emitter and the power receiver, the controller being configured to drive the signal emitter to emit vibrations in a predetermined sequence.

7. The marker of claim 5, the locator further including a power source, the power source being configured to store power received via the power receiver and communicate the stored power to the signal emitter.

8. The marker of claim 1, the locator including a piezoelectric crystal.

9. The marker of claim 1, the locator being configured to emit vibrations in the presence of ultrasonic radiation.

10. The marker of claim 1, the locator being configured to emit vibrations continuously in the presence of ultrasonic radiation.

11. The marker of claim 7, the locator being configured to emit vibrations imperceptible to humans.

12. The marker of claim 1, the locator being configured to emit vibrations of 25 Megahertz to 30 Megahertz.

13. The marker of claim 1, the locator being entirely enclosed by the carrier.

14. The marker of claim 1, the marker element including one or more legs, each leg of the one or more legs extending from an outer surface of the carrier, the locator being configured to communicate the vibrations through at least one leg of the one or more legs.

15. The marker of claim 1, the carrier being bioabsorbable, the marker element and the locator being permanent.

16. A system for locating a biopsy site, the system comprising:
(a) an ultrasound transducer; and
(b) a biopsy site marker, the marker including:
(i) a marker element, and
(ii) a locator proximate the marker element, the ultrasound transducer being configured to transmit ultrasonic radiation through tissue, the locator being configured to be excited by the ultrasonic radiation transmitted by the ultrasound transducer, thereby producing vibrations.

17. The system of claim 16, the locator being configured to resonate with the ultrasonic radiation transmitted by the ultrasound transducer.

18. The system of claim 16, the locator being coupled to a portion of the marker element, the marker element being configured to transmit the vibrations produced by the locator.

19. The system of claim 16, the marker further including a bioabsorbable carrier, the carrier enclosing the locator and at least a portion of the marker element.

20. A method for locating a biopsy site, the method comprising:
(a) inserting a portion of a marker delivery device into tissue to position a deployment opening of a cannula of the marker delivery device at the biopsy site;
(b) actuating a push rod of the marker delivery device to project a biopsy site marker through the deployment opening of the cannula of the marker delivery device and into the biopsy site; and
(c) inducing a vibration from at least a portion of the marker.

21.-23. (canceled)
